(19) United States
(12) Patent Application Publication
(10) Pub. No.: US 2025/0228499 A1
(43) Pub. Date: Jul. 17, 2025

(54) DEFIBRILLATION PROTECTION FOR MEDICAL EQUIPMENT SENSING CIRCUITS

(71) Applicant: Boston Scientific Scimed, Inc., Maple Grove, MN (US)

(72) Inventors: Szymon Rzeszowski, Minneapolis, MN (US); Michael P. Schrom, Forest Lake, MN (US)

(73) Assignee: Boston Scientific Scimed, Inc., Maple Grove, MN (US)

(21) Appl. No.: 19/014,943

(22) Filed: Jan. 9, 2025

Related U.S. Application Data
(60) Provisional application No. 63/620,270, filed on Jan. 12, 2024.

(51) Int. Cl. A61B 5/00; A61B 17/00; A61B 17/3203; A61N 1/39

(52) U.S. Cl. CPC A61B 5/7217; A61B 17/32037; A61B 2017/00026; A61N 1/3904

(57) ABSTRACT

A medical apparatus includes a therapy component for performing a therapeutic procedure on the patient. The therapy component includes sensing circuitry for sensing electrical signals from one or more sensing electrodes of the therapy component that are placed on and/or in the patient. With the medical apparatus powered on, a determination is made as to when the therapy component is actively engaged in performing the therapeutic procedure on the patient. When the therapy component is actively engaged in performing the therapeutic procedure on the patient, the one or more sensing electrodes of the therapy component are electrically connected to the sensing circuitry. When the therapy component is determined to not be actively engaged in performing the therapeutic procedure on the patient, the one or more sensing electrodes of the therapy component are electrically isolated from the sensing circuitry.

### CROSS-REFERENCE TO RELATED APPLICATIONS

This application claims the benefit of priority under 35 U.S.C. § 119 of U.S. Provisional Application No. 63/620,270, filed Jan. 12, 2024, the entire disclosure of which is hereby incorporated by reference.

### TECHNICAL FIELD

The disclosure is directed to medical equipment, and more particularly to defibrillation protection for medical equipment sensing circuits.

### BACKGROUND

Medical equipment often includes sensing circuitry that is used to sense physiological and/or other electrical signals in or on a patient's body. Such sensing circuits can be damaged by a defibrillation shock event that is applied to the patient body by a defibrillator. Some defibrillators have an in-built defibrillation protection circuit that is activated when the defibrillator is actively delivering defibrillation shock therapy to help protect the sensing circuits of the defibrillator from the defibrillation shock event delivered by the defibrillator. In many instances, the sensing circuits with defibrillator protection may be measuring signals that represent higher impedance measurements (e.g. greater than 100 Kilo-Ohms), such as EKG signals.

What would be desirable is a protection circuit for medical equipment that protects the medical equipment from electrical surges from a defibrillator that is separate from the medical equipment being protected by the protection circuit. What would also be desirable is a defibrillation protection circuit suitable for protecting sensing circuits that are configured to sense signals that represent low impedance measurements (e.g. less 50 Kilo-Ohms), such as signals indicative of blood clots or the like during a thrombectomy procedure, without negatively impacting the performance of any external defibrillator.

### SUMMARY

This disclosure provides design, material, manufacturing method, and use alternatives for medical devices. An example may be found in a method for operating a medical apparatus that is configured to perform a therapeutic procedure on a patient. The medical apparatus includes a therapy component for performing the therapeutic procedure on the patient, the therapy component includes one or more sensing electrodes that are adapted to be placed on and/or in the patient and sensing circuitry for sensing electrical signals from the one or more sensing electrodes. The method includes, with the medical apparatus powered on, determining when the therapy component is actively engaged in performing the therapeutic procedure on the patient. When the therapy component is determined to be actively engaged in performing the therapeutic procedure on the patient, the method includes having the one or more sensing electrodes of the therapy component electrically connected to the sensing circuitry of the therapy component. When the therapy component is determined to not be actively engaged in performing the therapeutic procedure on the patient, the method includes having the one or more sensing electrodes of the therapy component electrically isolated from the sensing circuitry of the therapy component.

Alternatively or additionally, when the therapy component is determined to not be actively engaged in performing the therapeutic procedure on the patient, each of the one or more sensing electrodes may be electrically isolated from the sensing circuitry by a respective relay in an open state.

Alternatively or additionally, when the therapy component is determined to be actively engaged in performing the therapeutic procedure on the patient, each of the one or more sensing electrodes may be electrically connected to the sensing circuitry by the respective relay in a closed state.

Alternatively or additionally, the method may further include filtering each of the electrical signals that is conducted through the respective relay in the closed state before the respective electrical signal reaches the sensing circuitry.

Alternatively or additionally, each of the respective relays may be a normally open relay that is switched to its closed state by passing a control current though a relay coil of the relay.

Alternatively or additionally, each of the respective normally open relays may be a high voltage relay rated for at least 5,000 volts.

Alternatively or additionally, the method may further include closing one or more normally open relays when the therapy component is determined to be actively engaged in performing the therapeutic procedure on the patient, wherein the one or more normally open relays, when closed, electrically connect the one or more sensing electrodes to the sensing circuitry. The method may further include opening the one or more normally open relays when the therapy component is determined to not be actively engaged in performing the therapeutic procedure on the patient, wherein the one or more normally open relays, when open, electrically isolate the one or more sensing electrodes from the sensing circuitry.

Alternatively or additionally, one or more of the electrical signals may be low impedance measurement signals measuring an impedance of less than 10K ohms.

Alternatively or additionally, one or more of the low impedance measurement

signals may correspond to a clot impedance measurement signal and/or a blood coagulation measurement signal.

Alternatively or additionally, the medical apparatus may include a thrombectomy apparatus and the therapeutic procedure may include a thrombectomy procedure.

Alternatively or additionally, the method may further include activating a switch to switch between actively engaging the therapy component in performing the therapeutic procedure on the patient and not actively engaging the therapy component in performing the therapeutic procedure on the patient.

Alternatively or additionally, the switch may be a manually operated switch that is manually operated by an operator of the medical apparatus.

Alternatively or additionally, the switch may be operated by a wired or wireless control signal provided by the medical apparatus.

Another example may be found in a medical apparatus for performing a therapeutic procedure on a patient. The medical apparatus includes a therapy component for performing the therapeutic procedure on the patient, the therapy component including sensing circuitry for sensing electrical signals from one or more sensing electrodes of the therapy component that are placed on and/or in the patient. The medical apparatus includes a control switch for switching between actively engaging the therapy component in performing the therapeutic procedure on the patient and disengaging the therapy component from actively performing the therapeutic procedure on the patient. The medical apparatus includes a sensing circuit protection switch operatively coupled between the one or more sensing electrodes and the sensing circuitry of the therapy component. The sensing circuit protection switch is configured to electrically connect the one or more sensing electrodes to the sensing circuitry when the control switch actively engages the therapy component in performing the therapeutic procedure on the patient, and to electrically disconnect the one or more sensing electrodes from the sensing circuitry when the control switch disengages the therapy component from performing the therapeutic procedure on the patient.

Alternatively or additionally, the sensing circuit protection switch may include one or more relays for selectively connecting the one or more sensing electrodes with the sensing circuitry of the therapy component when in a closed state and selectively isolating the one or more sensing electrodes from the sensing circuitry of the therapy component when in an open state.

Alternatively or additionally, the one or more relays may be switched between the open state and the closed state based on a position of the control switch.

Another example may be found in a defibrillation protection device for a medical apparatus. The defibrillation protection device may include a housing. One or more signal inputs are accessible from outside of the housing and are for receiving one or more low impedance measurement signals from one or more sensing electrodes that measure at an impedance of less than 10K ohms. One or more signal outputs are accessible from outside of the housing and are for passing the one or more low impedance measurement signals to a sensing circuit of the medical apparatus. The defibrillation protection device includes a control input and a defibrillation protection circuit housed that is by the housing and that is operatively coupled between the one or more signal inputs and the one or more signal outputs. The defibrillation protection circuit is configured to electrically connect the one or more signal inputs to the one or more signal outputs when the control input is in a first state and to electrically disconnect the one or more signal inputs from the one or more signal outputs when the control input is in a second state.

Alternatively or additionally, the control input may correspond to a manually operated switch.

Alternatively or additionally, the control input may correspond to a wired or wireless signal provided by the medical apparatus indicating when the medical apparatus is actively engaged in performing a therapeutic procedure on a patient.

Alternatively or additionally, the defibrillation protection circuit may include one or more relays that are closed when the control input is in the first state to electrically connect the one or more signal inputs to the one or more signal outputs, and are opened when the control input is in the second state to electrically disconnect the one or more signal inputs from the one or more signal outputs.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of a portion of the thrombectomy system of FIG. 1;

FIG. 3 is a schematic view of a portion of an illustrative thrombectomy system;

FIG. 4 is a schematic block diagram showing an illustrative medical apparatus;

FIG. 5 is a flow diagram showing an illustrative method that may be carried using the illustrative medical apparatus of FIG. 4;

FIG. 6 is a schematic block diagram showing an illustrative defibrillation protection device;

FIG. 7 is a schematic diagram showing an illustrative implementation of a sensing circuit protection switch as shown in FIG. 3; and

FIG. 8 is a schematic diagram showing an illustrative electrical circuit of a sensing circuit protection switch as shown in FIG. 3.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

A defibrillation protection circuit for medical equipment is disclosed. In some cases, the defibrillation protection circuit is deactivated (e.g. allows the sensing signals to pass through) when the medical equipment is engaged in delivering therapy to a patient, and activated (e.g. prevents the sensing signals from passing through) when the medical equipment is not engaged in delivering therapy to the patient. In some cases, the defibrillation protection circuit is configured to protect sensing circuits that sense signals that represent low impedance measurements (e.g. less than 50 Kilo-Ohms), such as signals indicative of blood clot, blood coagulation and/or the like during a thrombectomy or other medical procedure. It is contemplated that the defibrillation protection circuit may be used in conjunction with a wide variety medical equipment including, for example, thrombectomy systems, intracardiac signal detection/mapping, ECG, Electrical Impedance Myography (EIM), electrical impedance tomography, bipolar RF ablation, irreversible electroporation, as well as others. To provide a concrete example, use of the defibrillation protection circuit in a thrombectomy system is described below.

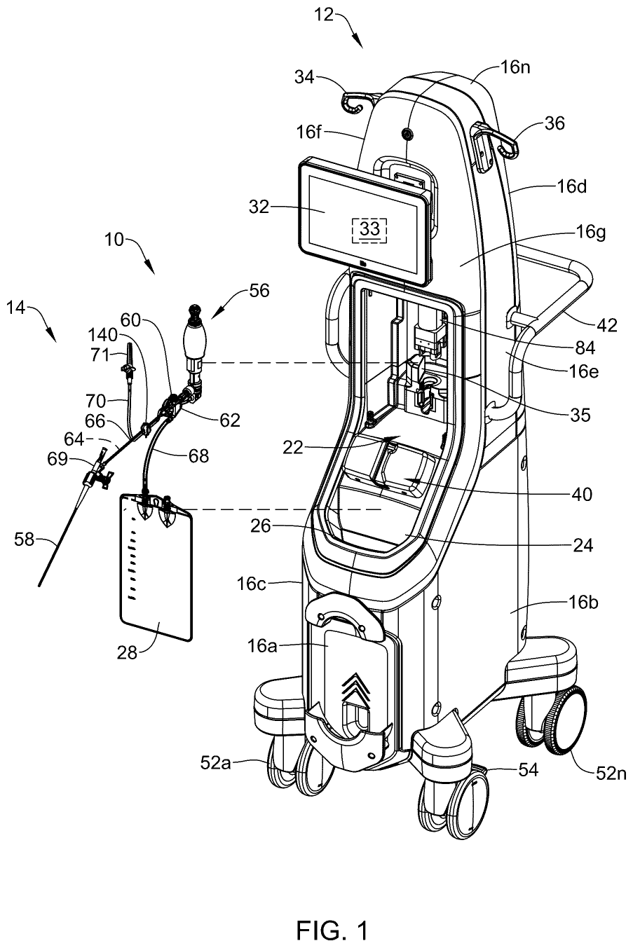

Thrombectomy catheters and systems may be used to remove thrombus, plaques, lesions, clots, etc. from veins or arteries. FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a disposable single use device in which a new pump/catheter assembly 14 may be used with the console 12 for each medical procedure. The console 12 may include a housing enclosing the internal structure of the console 12. Shown on the console 12 are a plurality of removable panels 16a-16n about and along the console 12 enclosing the internal structure of the console 12. An illustrative console 12 is described in commonly assigned U.S. Pat. No. 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies (not explicitly shown) which open to expose the interior of the console 12 to provide access to a carriage assembly 22 and close during use of the pump/catheter assembly 14. Illustrative loading bay doors and assemblies are described in commonly assigned U.S. Patent Application No. 63/452,517, titled THROMBECTOMY SYSTEM WITH LOADING BAY DOORS, the disclosure of which is hereby incorporated by reference.

The console 12 may include a catch basin or drip tray 24 for collecting fluid leakage from the components of the pump/catheter assembly 14. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. The effluent waste tube 68 may also be positioned in the roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on a panel 16g, to selectively position the carriage assembly 22 inwardly or outwardly. In other instances, the carriage assembly 22 may be positioned or moved using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen.

Saline bag hooks 34 and 36 may extend through the panels 16e and 16f to hang saline bags therefrom. The console 12 may include a handle 42 as well as a plurality of wheels 52a-52n and brake pedals 54 for wheel lockage to assist in maneuvering the console 12 by medical personnel.

In FIG. 1, the pump/catheter assembly 14 is shown detached from the console 12. The pump/catheter assembly 14 includes an inflow pump 56 and a thrombectomy device 58. In some embodiments, the inflow pump 56 may be configured to provide fluid inflow through the thrombectomy device 58. During use, a portion of the pump/catheter assembly 14 may be secured within a portion of the console 12. In some embodiments, the pump/catheter assembly 14 may include a bubble trap 60 attached to the inflow pump 56, a connection manifold assembly 62 connected to the bubble trap 60, a fixture 140, an effluent return tube 66 connected between the connection manifold assembly 62 and the thrombectomy device 58, a high-pressure fluid supply tube 64 attached between the output of the inflow pump 56 and the thrombectomy device 58 which may be coaxially arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag (e.g., a saline bag) (not explicitly shown) to the connection manifold assembly 62. The fluid supply tube 70 may be in fluid communication with the interior of the bubble trap 60 to provide fluid from the fluid supply bag to the inflow pump 56 and then to the thrombectomy device 58 through the high-pressure fluid supply tube 64.

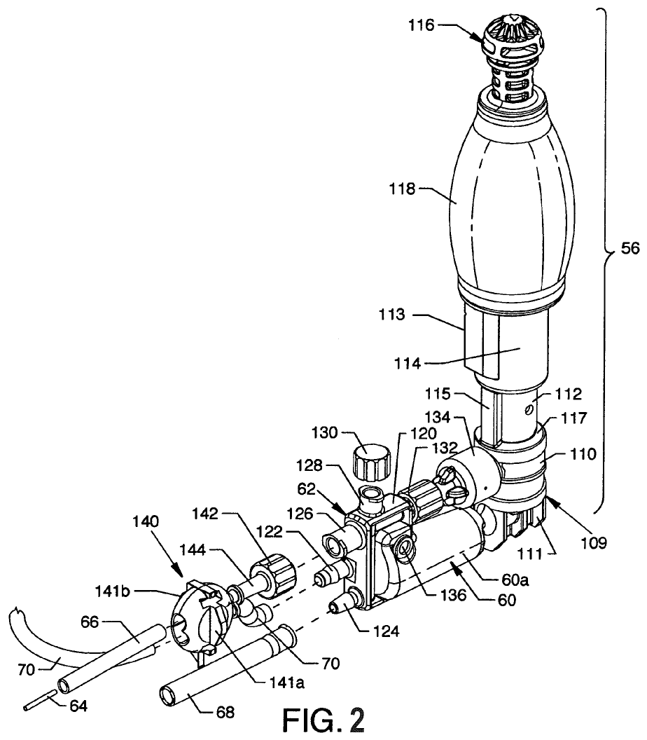

The console 12 may include a reciprocating linear actuator 84 configured to engage a pump piston head 116 (see, for example, FIG. 2) of the inflow pump 56 when the inflow pump 56 is engaged with the carriage assembly 22. The reciprocating linear actuator 84 may be disposed within the interior of the console 12 and may be aligned with the pump piston head 116 (e.g., FIG. 2) when the inflow pump 56 is disposed within the interior of the console 12. The reciprocating linear actuator 84 may be actuated such that reciprocating (e.g., up and down) strokes of the reciprocating linear actuator 84 drive the inflow pump 56 in response to activation of a user activation switch (not explicitly shown) or a control command from a controller 33. In some embodiments, the user activation switch may be a foot switch. In some embodiments, when the user activation switch is depressed, the reciprocating linear actuator 84 and/or the inflow pump 56 is activated and/or runs, and when the user activation switch is released, the reciprocating linear actuator 84 and/or the inflow pump 56 is stopped and/or ceases operation.

The console 12 may include a controller 33 in electronic communication with the user interface 32, the reciprocating linear actuator 84, the inflow pump 56, and/or the thrombectomy device 58. In some cases, the controller 33 may be a part of or otherwise incorporated into the user interface 32. In some embodiments, the console 12 and/or the controller 33 may include a data acquisition device 35. In some embodiments, the data acquisition device 35 may be disposed within the interior of the console 12. For example, the data acquisition device 35 may be positioned on or near the carriage assembly 22. In some embodiments, the data acquisition device 35 may be configured for wireless communication. Other configurations are also contemplated. The data acquisition device 35 may be a radiofrequency identification reader and/or a barcode reader. Other types of data acquisition devices 35 may be used, as desired. In some embodiments, the user activation switch may be in electronic communication with the console 12 and/or the controller 33. In some embodiments, the user activation switch may be in electronic communication with the console 12 and/or the controller 33 via a wire or cable. In some embodiments, the user activation switch may be in electronic communication with the console 12 and/or the controller 33 wirelessly.

FIG. 2 is a partially exploded perspective view of several components of the pump/catheter assembly 14 (e.g., FIG. 1) generally including the inflow pump 56, the bubble trap 60, the connection manifold assembly 62, and the fixture 140. The inflow pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 (e.g., FIG. 1) to retain the inflow pump 56 within and/or in engagement with the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example.

In some embodiments, a data plate 113 may also be included on the pump/catheter assembly 14, such as on the top body 114 for example, for the inclusion of a barcode, an radiofrequency identification (RFID) tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof. In at least some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain specifications and/or operational parameters associated with the pump/catheter assembly 14, the thrombectomy device 58, the inflow pump 56, etc., and/or components thereof. In some embodiments, the data acquisition device 35 (e.g., FIG. 1) may be configured to communicate with the data plate 113 (e.g., the barcode, the RFID tag, the data storage chip, etc.), the pump/catheter assembly 14, the thrombectomy device 58, the inflow pump 56, etc. to obtain identifying information related thereto which is associated with specifications and/or operational parameters stored in the memory of the user interface 32 and/or the controller 33. The identifying information may be used by the user interface 32 and/or the controller 33 to access the specifications and/or operational parameters associated with the pump/catheter assembly 14, the thrombectomy device 58, the inflow pump 56, etc. in use that is stored in the memory.

In some embodiments, the inflow pump 56 may include a hemispherically-shaped pump piston head 116 having a flexible boot 118 connected to and extending between the top body 114 and the pump piston head 116. In some instances, the lower portion 111 of the base 109 may serve as a mount for a first end of the bubble trap 60.

The connection manifold assembly 62 may be secured directly to a second end of the bubble trap 60 and in some instances may include a bracket 120 including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include two mating halves of which a first bubble trap half 60a is shown. A hydrophobic filter 136 may be included at the upper forward region of the first bubble trap half 60a. In some embodiments, a second hydrophobic filter may be included on the second bubble trap half (not explicitly shown) which opposes the hydrophobic filter 136 on the first bubble trap half 60a.

The fixture 140, and components associated therewith, assists in support and connection of the effluent return tube 66 to the effluent return port 126 by a connector 142 combined continuously with a connection tube 144, and also assists in support, passage, and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting the roller pump 40 (e.g., FIG. 1) provided with the console 12, such as located in the carriage assembly 22 or adjacent thereto.

In some embodiments, the effluent waste tube 68 may be positioned within and/or through the roller pump with the effluent collection bag 28 (e.g., FIG. 1) connected to the effluent waste tube 68. The effluent collection bag 28 may be suitably positioned for collecting effluent during the procedure. Pump rollers of the roller pump 40 may engage the effluent waste tube 68 to control effluent fluid flow through the effluent waste tube 68 from the effluent outlet port 124 to the effluent collection bag 28. In some embodiments, other pump types and/or configurations may be used in place of the roller pump.

At an appropriate time, the thrombectomy device 58 may be subjected to a priming procedure to purge the thrombectomy device 58 of any air. For example, the tip of the thrombectomy device 58 may be placed in a bowl of sterile saline, or other fluid, and the inflow pump 56 may be operated by action of the reciprocating linear actuator 84 (such as by activating and/or depressing the user activation switch) to prime the thrombectomy device 58. Thereafter, medical personnel may insert the thrombectomy device 58 into the vasculature of the patient, and operation of the thrombectomy system 10 incorporating the user interface 32 and the user activation switch (not shown) can begin, as desired. The reciprocating linear actuator 84 is actuated according to the operating parameters to influence fluid inflow pressures, pump speed, flow rates, and the like to operate the inflow pump 56 to deliver pressurized fluid to the thrombectomy device 58 via the high-pressure fluid supply tube 64 residing in the effluent return tube 66. Supply fluid is routed through the bubble trap 60, may be pressurized by the inflow pump 56, and is routed through the high-pressure fluid supply tube 64 to the thrombectomy device 58 for use in a thrombectomy or other related procedure. Effluent may be returned through the effluent return tube 66 to the connection manifold assembly 62 for collection in the effluent collection bag 28 (e.g., FIG. 1) through the effluent waste tube 68, which may be controlled by the roller pump 40.

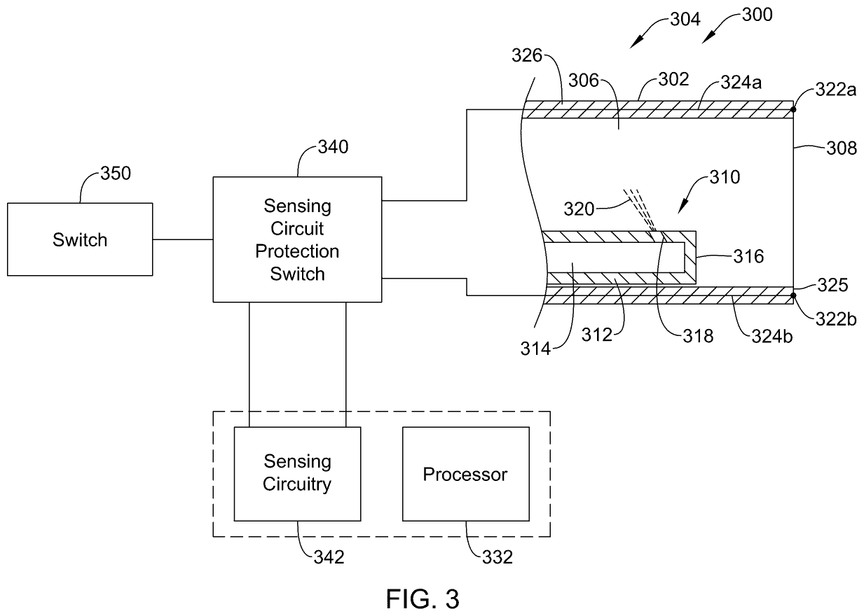

FIG. 3 illustrates a cross-sectional view of the distal end region of an example thrombectomy catheter 300. The thrombectomy catheter 300 may be considered as being an example of the thrombectomy device 58. In some instances, the thrombectomy catheter 300 may include a tubular member or catheter body 302 extending from a proximal end region (not explicitly shown) configured to remain outside the body to a distal end region 304. The catheter body 302 may be one illustrative example of, or be in fluid communication with, the effluent return tube 66 of the thrombectomy device 58 described above. A lumen 306 may extend from the proximal end region to the distal end region 304 of the catheter body 302. The catheter body 302 may terminate at a distally facing distal opening 308 at the distal end of the catheter body 302. In some instances, the distal opening 308 may be in a plane that extends generally orthogonal to a longitudinal axis of the catheter body 302. In other instances, the distal opening 308 may be in a plane that extends generally oblique to a longitudinal axis of the catheter body 302.

The thrombectomy catheter 300 may further include a high-pressure fluid supply tube 310. The high-pressure fluid supply tube 310 may be one illustrative example of, or be in fluid communication with, the high-pressure fluid supply tube 66 of the thrombectomy device 58 described above. The high-pressure fluid supply tube 310 may be disposed within and extend through the lumen 306 of the catheter body 302. The high-pressure fluid supply tube 310 may include a supply tube wall 312 defining a lumen or fluid pathway 314 extending therethrough. In at least some instances, the high-pressure fluid supply tube 310 may have a closed distal end 316. Because of this, fluid may be able to pass distally through the fluid pathway 314 but does not exit the distal end. The high-pressure fluid supply tube 310 may extend along a length of the catheter body 302 with the distal end 316 located within the lumen 306 of the catheter body 302 proximal to the distal opening 308 at the distal end of the catheter body 302. A proximal end of the high-pressure fluid supply tube 310 may be in fluid communication with the pump 56 described herein, to provide high-pressure fluid to the fluid pathway 314 of the high-pressure fluid supply tube 310.

The thrombectomy catheter 300 may include one or more jet orifices 318 which may be defined along the supply tube wall 312. While only one jet orifice is depicted in FIG. 3, it can be appreciated that the supply tube wall 312 may include one, two, three, four, five, six, or more jet orifices 318. Infusion of motive fluid through the lumen 314 of the supply tube wall 312 may result in fluid being jetted through the jet orifices (e.g., the one or more jet orifices 318) and the generation of a proximally directed aspiration force through the catheter body 312. Further, entrainment material may enter the distal opening 308 and then may be urged proximally by momentum transfer. As the mixture of jetted motive fluid 320 and entrainment material moves proximally, the material may sequentially approach a number of jet orifices (e.g., the one or more jet orifices 318) positioned along the supply tube wall 312. Upon interaction with the jetted motive fluid 320 from each individual jet orifice 318, the momentum in the entrainment material mixture may increase, and the thrombogenic material may more readily flow proximally through the catheter body 302 for removal.

In some instances, the thrombectomy catheter 300 may include a first electrode 322a positioned within the wall 326 of the catheter body 302 and a second electrode 322b positioned within the wall 326 of the catheter body 302. It can be appreciated that the first electrode 322a may be radially, axially and/or circumferentially spaced (e.g., offset, etc.) from the second electrode 322b at any position (e.g., longitudinal position) along the catheter body 302. As an example, the first electrode 322a and the second electrode 322b may be positioned along a distal facing surface 325 of the catheter body 302. In some cases, the first electrode 322a and/or the second electrode 322b may be positioned axially along any portion of the distal end region 304 of the catheter body 302. For example, the first electrode 322a and/or the second electrode 322b may be positioned at any location proximally of the distal end of the catheter body 302 (e.g., the first electrode 322a and/or the second electrode 322b may be positioned within the wall 326 of the proximal body 302 at any position proximal of the distal facing surface 325 of the catheter body 302). In some cases, the first electrode 322a may be attached to a first wire 324a. The first wire 324a may be positioned within the wall 326 of the catheter body 302. In some instances, the second electrode 322b may be attached to a second wire 324b. The second wire 324b may also be positioned within the wall 326 of the catheter body 302.

While the first electrode 322a and the second electrode 322b are shown positioned within the wall 326 of the catheter body 302, it can be appreciated that the first electrode 322a, the second electrode 322b or both the first electrode 322a and the second electrode 322b may be positioned substantially flush with an inner surface of the catheter body 302. Further, it can be appreciated that the first electrode 322a, the second electrode 322b or both the first electrode 322a and the second electrode 322b may be positioned substantially flush with an outer surface of the catheter body 302. Further, it can be appreciated that the first wire 324a, the second wire 324b or both the first wire 324a and the second wire 324b may be positioned substantially flush with an inner surface of the catheter body 302. Further, it can be appreciated that the first wire 324a, the second wire 324b or both the first wire 324a and the second wire 324b may be positioned substantially flush with an outer surface of the catheter body 302.

FIG. 3 further illustrates that the first wire 324a and the second wire 324b may extend from the first electrode 322a, 322b, respectively, through the wall 326 of the catheter body and eventually operatively coupled to a sensing circuitry 342. In some instances, the sensing circuitry 342 may be separate from a processor 332. In some instances, the sensing circuitry 342 may be considered to be part of the processor 332, as indicated by the dashed line around the processor 332 and the sensing circuitry 342. The thrombectomy system may include an activation switch 350 that may be used to actuate and de-actuate the reciprocating linear actuator 84 to drive the inflow pump 56. The switch 350 may be a foot-operated switch, for example, or a hand-held switch. The switch 350 may be wired to the thrombectomy system, or may be coupled to the thrombectomy system via a wireless connection. In some cases, while the switch 350 is depressed, the reciprocating linear actuator 84 and/or the inflow pump 56 are activated and/or run, and when the switch 350 is released, the reciprocating linear actuator 84 and/or the inflow pump 56 are stopped and/or cease operation.

In some instances, the switch 350 may be operatively coupled to a sensing circuit protection switch 340. The sensing circuit protection switch 340 may be an example of a defibrillation protection circuit for the sensing circuitry 342. The sensing circuit protection switch 340 may be configured to selectively connect and disconnect the electrodes 322a and 322b from the sensing circuitry 342 in order to protect the sensing circuitry 342 from power surges (e.g. defibrillation shock therapy) that could result when other electrical devices such as a defibrillator are actuated within or on the patient. The switch 350 may be coupled to the sensing circuit protection switch 340 so that the sensing circuit protection switch 340 is able to determine whether the switch 350 is open or closed, and to control the sensing circuit protection switch 340 based on the state of the switch 350. In some cases, the sensing circuit protection switch 340 is deactivated (e.g. allows the sensing signals from electrodes 322a and 322b to pass) when the switch 350 is closed and the thrombectomy system 10 is engaged in delivering therapy to the patient, and activated (e.g. prevents the sensing signals from electrodes 322a and 322b to pass) when the thrombectomy system 10 is not engaged in delivering therapy to the patient. In some cases, the sensing circuit protection switch 340 is configured to introduce a low impedance when the sensing circuit protection switch 340 is deactivated (e.g. allows the sensing signals from electrodes 322a and 322b to pass) so as to allow the sensing circuitry 342 to make low impedance measurements (e.g. less than 50 Kilo-Ohms) such as measurements indicative of blood clots or the like during a thrombectomy procedure.

The thrombectomy system 10 may be configured to send an electrical signal from the processor 332 to the first electrode 322a via the first wire 324a, whereby the electrical signal passes from the first electrode 322a, through the bodily substance present at the distal end region of the catheter body 302 (e.g., through the bodily substance positioned between the first electrode 322a and the second electrode 322b), whereby the electrical signal is then received by the second electrode 322b. It can further be appreciated that the electrical signal may then be passed from the second electrode 322b back to the sensing circuitry 342 through the second wire 324b. The sensing circuitry 342 may be configured to determine the impedance of this electrical path, which includes the impedance of the bodily substance positioned adjacent to the first electrode 322a and the second electrode 322b. The impedance of the bodily substance positioned adjacent to the first electrode 322a and the second electrode 322b may be less than 50 Kilo-Ohms, less than 20 Kilo-Ohms, less than 10 Kilo-Ohms, less than 1 Kilo-Ohm, and/or in a range between 100 Ohms and 1 Kilo-Ohms. Because this measurement may be a low impedance measurement (less than 50 Kilo-Ohms), the impedance introduced by the sensing circuit protection switch 340 must be low so as to not obscure, interfere with and/or otherwise significantly reduce the accuracy of the low impedance measurement of the bodily substance positioned adjacent to the first electrode 322a and the second electrode 322b.

In some instances, the sensing circuitry 342 may identify a measure of the impedance (resistive and/or reactive impedance) of the bodily substance positioned between the first electrode 322a and the second electrode 322b. Further, the sensing circuitry 342 may be configured to compare a measured impedance to an approximate, preset (e.g., preprogrammed) ranges for the impedance of blood versus the impedance of a clot (e.g., plaque, thrombus, etc.). The preset (e.g., preprogrammed) ranges for the impedance of blood versus the impedance of a clot (e.g., plaque, thrombus, etc.) may be stored in a memory accessible by the sensing circuitry 342. In other examples, the ranges for the impedance of blood versus the impedance of a clot (e.g., plaque, thrombus, etc.) may be input into the system 10 by a clinician via a touchpad on the console 330. In other words, the sensing circuitry 342 may be configured to compare the impedance sensed between the first electrode 322a and the second electrode 322b to a preprogrammed value of a bodily substance positioned adjacent to the first electrode 322a and the second electrode 322b.

It can be further appreciated that the system 10 may be configured such that when the measured impedance is within a range which indicates the tip of the catheter 300 is adjacent to a clot (e.g., plaque, thrombus, etc.), the sensing circuitry 342 and/or the processor 332 may send a signal activating an electrical circuit that starts the pump 56. In some cases, the sensing circuitry 342 and/or the processor 332 may instead send a signal to inform the user to start the pump 56. As discussed herein, starting the pump 56 may cause infusion of motive fluid through the lumen 314 of the supply tube wall 312, resulting in fluid being jetted through the jet orifices (e.g., the one or more jet orifices 318) and the generation of a proximally directed aspiration force through the catheter body 312 to remove the clot (e.g., plaque, thrombus, etc.).

The sensing circuit protection switch 340 may be configured to protect low impedance measurement systems so as to not significantly interfere with the low impedance measurements. The thrombectomy system 10 is an example of a medical device having electrical circuitry that may benefit from such low impedance measurements. However, other medical systems may also benefit including those that operate at a higher impedance include intracardiac signal detection and mapping, ECG (electrocardiogram), EIM (electrical impedance myography) and electrical impedance tomography. In some instances, ablation devices such as bipolar RF (radio frequency) ablation and irreversible electroporation may benefit from inclusion of the sensing circuit protection switch 340. Farapulse™, available commercially from Boston Scientific, is an example of an electroporation device.

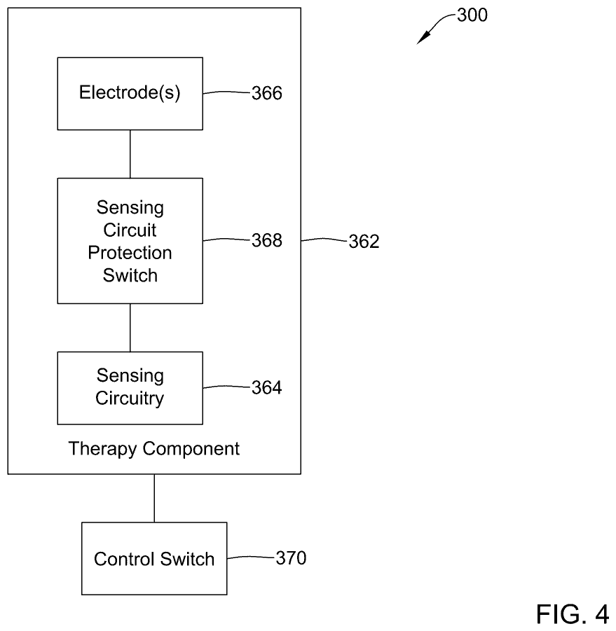

FIG. 4 is a schematic block diagram showing an illustrative medical apparatus 360. The illustrative medical apparatus 360 may represent the thrombectomy system 10, the thrombectomy catheter 300, or any of a variety of different medical equipment that may benefit from inclusion of a protective circuit that is configured to protect sensitive sensing circuits from excessive electrical pulses resulting from, for example, a defibrillator firing. The illustrative medical apparatus 360 includes a therapy component 362 that is configured to carry out a therapeutic procedure on a patient. The therapy component 362 includes sensing circuitry 364 for sensing electrical signals from sensing electrode(s) 366 associated with the therapy component 362. The sensing electrode(s) 366 may be configured to be disposed inside of a patient. In some instances, the sensing electrode(s) 366 may be configured to be disposed on an exterior of a patient, such as on the patient's skin. The sensing circuitry 364 may be considered as an example of the sensing circuitry 342 shown in FIG. 3. A sensing circuit protection switch 368, which may be considered as an example of the sensing circuit protection switch 340 shown in FIG. 3, is disposed between the sensing electrode(s) 366 and the sensing circuitry 364. A control switch 370 may be used to actively engage, or alternatively, actively disengage the sensing circuit protection switch 368, the therapy component 362 and/or the medical apparatus 360, even while the medical apparatus 360 is itself powered on. The sensing circuit protection switch 368 may be considered an example of a defibrillation protection circuit.

In some instances, the control switch 370 is operatively coupled with the sensing circuit protection switch 368 so that the sensing circuit protection switch 368 is able to determine when the control switch 370 is in an open position and when the control switch 370 is in a closed position. In some instances, the sensing circuit protection switch 368 may be configured to electrically connect the sensing electrode(s) 366 with the sensing circuitry 364 when the therapy component 362 is actively engaged in performing a therapeutic procedure on the patient. In some instances, the sensing circuit protection switch 368 may be configured to electrically disconnect the sensing electrode(s) 366 from the sensing circuitry 364 when the therapy component 362 is not actively engaged in performing the therapeutic procedure on the patient. In some instances, the sensing circuit protection switch 368 may include one or more relays for selectively connecting the sensing electrode(s) 366 with the sensing circuitry 364 when in a closed state and selectively isolating the sensing electrode(s) 366 from the sensing circuitry 364 when in an open state. Relays provide a low impedance path between the sensing electrode(s) 366 and the sensing circuitry 364 when in the closed state, which can be useful when attempting to sense signals that represent low impedance measurements (e.g. less than 50 Kilo-Ohms), such as signals indicative of blood clots or the like during a thrombectomy procedure. In some cases, the one or more relays may be switched between the open state and the closed state based on a position of the control switch 370. The control switch 370 may be a foot switch, a hand switch and/or any other suitable switch. In some cases, the switch 370 can be activated (e.g., pressed and held down) by an operator of the medical apparatus 360 to activate the therapy component 362 of the medical apparatus 360 and deactivate (e.g. allow the sensing signals to pass through) the sensing circuit protection switch 368, and deactivated (e.g. released) by the operator of the medical apparatus 360 to deactivate the therapy component 362 of the medical apparatus 360 and activate (e.g. prevent the sensing signals from passing through) the sensing circuit protection switch 368.

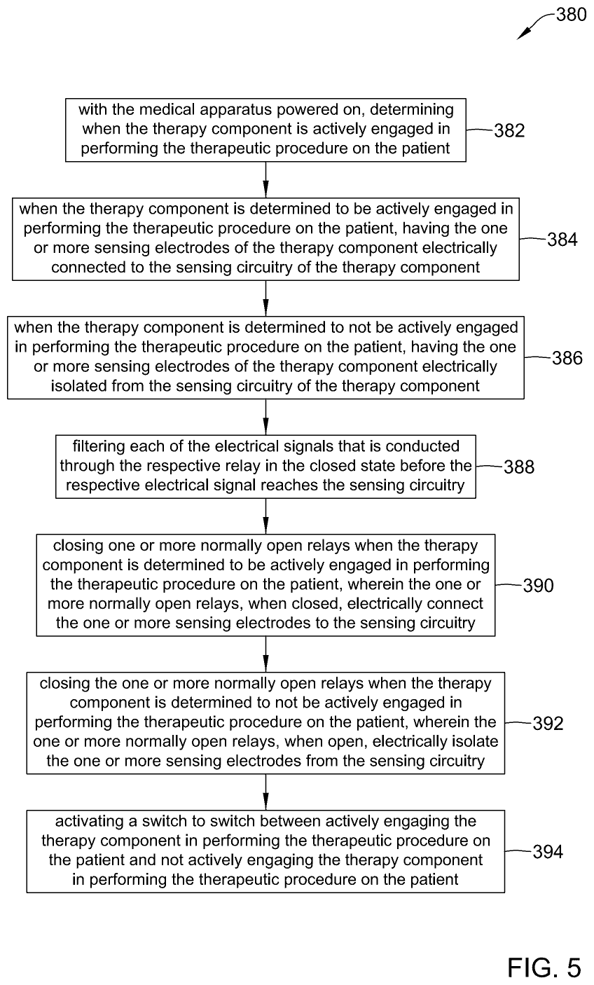

FIG. 5 is a flow diagram showing an illustrative method 380 that may be carried out in a medical apparatus (such as the medical apparatus 360). With the medical apparatus powered on, the illustrative method 380 includes determining when the therapy component is actively engaged in performing a therapeutic procedure on the patient, as indicated at block 382. When the therapy component is determined to be actively engaged in performing the therapeutic procedure on the patient, the one or more sensing electrodes of the therapy component are electrically connected to the sensing circuitry of the therapy component, as indicated at block 384. In some instances, each of the sensing electrode(s) may be electrically connected to the sensing circuitry by a respective relay in a closed state. When the therapy component is determined to not be actively engaged in performing the therapeutic procedure on the patient, the one or more sensing electrodes of the therapy component are electrically isolated from the sensing circuitry of the therapy component, as indicated at block 386. In some instances, each of the sensing electrode(s) may be electrically disconnected from the sensing circuitry by a respective relay in an open state.

In some instances, the method 380 may further include filtering each of the electrical signals that is conducted through the respective relay in the closed state before the respective electrical signal reaches the sensing circuitry, as indicated at block 388. In some cases, each of the respective relays may be a normally open relay that is switched to its closed state by passing a control current though a relay coil of the respective relay. As an example, each of the respective normally open relays may be a high voltage relay rated for at least 5,000 volts. In some instances, one or more of the electrical signals may be low impedance measurement signals measuring an impedance of less than 10,000 (10K) ohms. As an example, one or more of the low impedance measurement signals may correspond to a clot impedance measurement signal and/or a blood coagulation measurement signal. In some instances, the medical apparatus may be a thrombectomy apparatus and the therapeutic procedure may include a thrombectomy procedure.

In some instances, the method 380 may include closing one or more normally open relays when the therapy component is determined to be actively engaged in performing a therapeutic procedure on the patient, wherein the one or more normally open relays, when closed, electrically connect the one or more sensing electrodes to the sensing circuitry, as indicated at block 390. The method 380 may further include opening the one or more normally open relays when the therapy component is determined to not be actively engaged in performing the therapeutic procedure on the patient, wherein the one or more normally open relays, when open, electrically isolate the one or more sensing electrodes from the sensing circuitry, as indicated at block 392.

In some cases, the method 380 may include activating a switch to switch between actively engaging the therapy component to perform the therapeutic procedure on the patient and closing the respective relays, and not actively engaging the therapy component in performing the therapeutic procedure on the patient and opening the respective relays, as indicated at block 394. In some cases, the switch may be a manually operated switch that is manually operated by an operator of the medical apparatus. In some instances, the switch may be operated by a wired or wireless control signal provided by the medical apparatus.

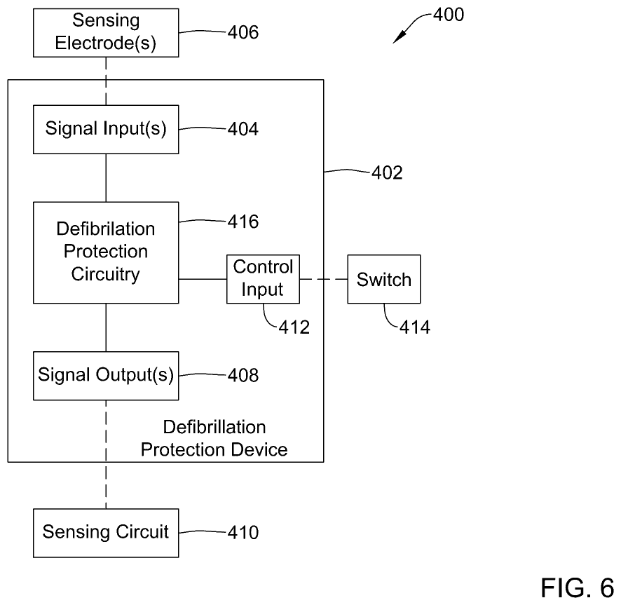

In some instances, an existing medical apparatus may be retrofitted to include the functionality of the sensing circuit protection switch. FIG. 6 is a schematic block diagram showing an illustrative defibrillation protection device 400 that may be utilized with any of a variety of different medical apparatuses. The defibrillation protection device 400 includes a housing 402. The defibrillation protection device 400 includes signal input(s) 404 that are accessible from outside of the housing 402. The signal input(s) 404 are configured to receive one or more low impedance measurement signals from sensing electrode(s) 406 that are exterior to the housing 402. In some cases, the low impedance measurement signals may measure an impedance of less than 10K ohms. The defibrillation protection device 400 includes signal output(s) 408 that are accessible from outside of the housing 402. The signal output(s) 408 are configured for passing the one or more low impedance measurement signals to a sensing circuit 410 that is exterior to the housing 402. A control input 412 is disposed within the housing 402, and may be accessible from outside of the housing 402 such that the control input 412 is able to receive a signal from a switch 414. The signal from the switch 414 may provide an indication of whether the switch 414 is in an open position or a closed position, for example. In some instances, the switch 414 connected to the control input 412 may be a manually operated switch. In some cases, the switch 414 connected to the control input 412 may be a wired or wireless signal provided by the medical apparatus indicating when the medical device is actively engaged in performing a therapeutic procedure on a patient.

A defibrillation protection circuit 416 is housed by the housing 402. The defibrillation protection circuit 416 is operatively coupled between the signal input(s) 404 and the signal output(s) 408 and is configured to electrically connect the signal input(s) 404 to the signal output(s) 408 when the control input is in a first state and to electrically disconnect the signal input(s) 404 from the signal output(s) 408 when the control input 412 is in a second state. In some instances, the defibrillation protection circuit 416 may include one or more relays, wherein the one or more relays are closed when the control input 412 is in the first state to electrically connect the signal input(s) 404 to the signal output(s) 408, and the one or more relays are opened when the control input 412 is in the second state to electrically disconnect or isolate the signal input(s) 404 from the signal output(s) 408.

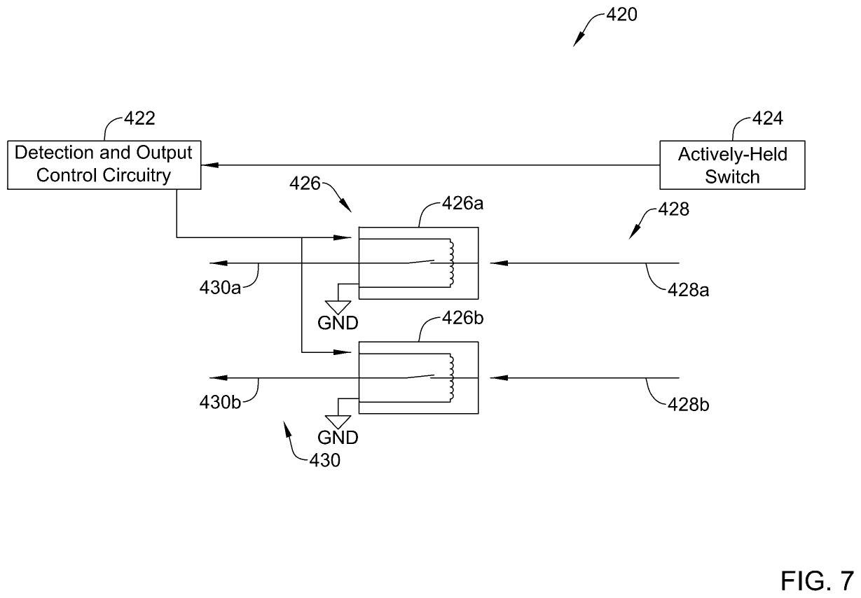

FIG. 7 is a schematic block diagram showing an illustrative implementation of a sensing circuit protection circuit 420 (such as the sensing circuit protection switch 340, the sensing circuit protection switch 368 or the defibrillation protection circuit 416). The sensing circuit protection circuit 420 includes detection and output control circuitry 422. An actively-held switch 424 may be used to selectively actuate or de-actuate a therapy of a medical apparatus, and in the example shown, is coupled to the detection and output control circuitry 422. The sensing circuit protection circuit 420 includes one or more switches 426, individually labeled as 426a and 426b. While two switches 426 are shown, this is merely illustrative, as the sensing circuit protection circuit 420 may include any number of switches as desired each corresponding to a sensing electrode. Each switch 426 may be a normally-open, high-isolation switch that is isolated when the actively-held switch 424 is not activated, or when the power is off, or a fault is present. The switches 426 are preferably low impedance when activated. For example, the switches 426 may be relays, transistors (e.g. power FETs), etc.

Each switch 426 is disposed between an input signal 428, individually labeled as 428a and 428b, and an output signal 430, individually labeled as 430a and 430b. The input signals 428 are incoming signals from sensing electrodes, for example. The output signals 430 are directed to sensing circuitry (not shown). In some instances, the output signals 430 may pass through additional defibrillation protective electronics before reaching the sensing circuitry (not shown), if desired.

During use, when the switches 426 are normally open relays, and when the actively-held switch 424 is pressed and held down by an operator indicating a therapy is being performed, the detection and output control circuitry 422 provides a control current to the coils of the normally open switches 426, which causes the normally open switches 426 to close and provide a low impedance path between respective input signals 428 and output signals 430. When the operator releases the actively-held switch 424, indicating a therapy is not being performed, the detection and output control circuitry 422 removes the control current to the coils of the normally open switches 426, which causes the normally open switches 426 to open and disconnect or isolate the input signals 428 from the output signals 430. When the switches 426 are normally closed switches, and when the actively-held switch 424 is pressed and held down by an operator indicating a therapy is being performed, the detection and output control circuitry 422 does not provide a control current to the coils of the normally closed switches 426, which causes the normally closed switches 426 to close and provide a low impedance path between respective input signals 428 and output signals 430. When the operator releases the actively-held switch 424, indicating a therapy is not being performed, the detection and output control circuitry 422 provides a control current to the coils of the normally closed switches 426, which causes the normally closed switches 426 to open and disconnect or isolate the input signals 428 from the output signals 430. In some cases, the switches 426 may be latching relays, and the detection and output control circuitry 422 may be configured to provide a temporary state setting current to the coils of the relays, in a desired polarity, to set the state of the latching relays to a desired open or closed state according to the action of the actively-held switch 424. The latching relay remains in that latched state until the state is again changed by application of a subsequent temporary state setting current.

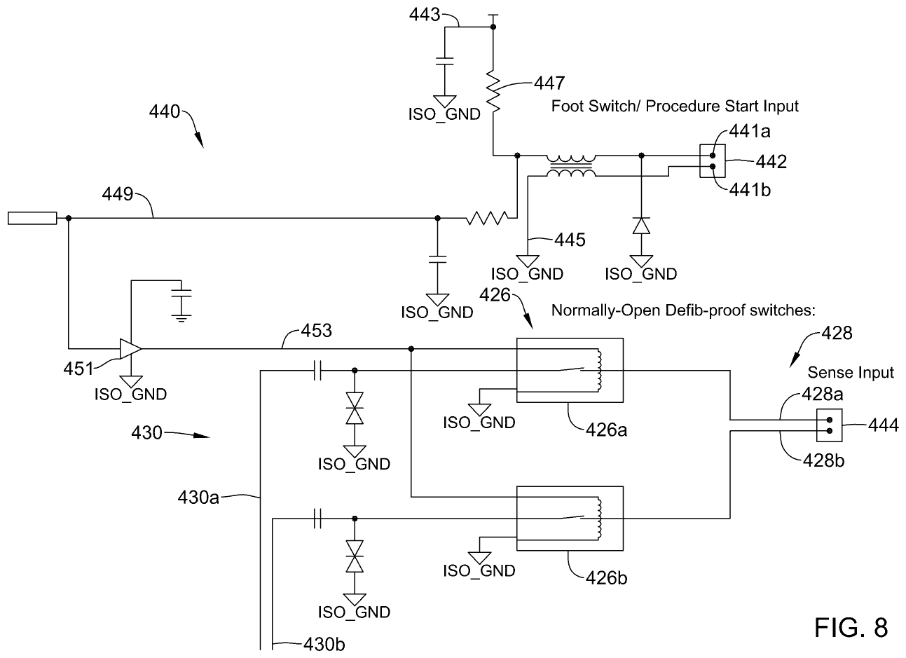

FIG. 8 is a schematic block diagram showing an illustrative implementation of a sensing circuit protection circuit 440 (such as the sensing circuit protection switch 340, the sensing circuit protection switch 368 or the defibrillation protection circuit 416). The sensing circuit protection circuit 440 includes an input 442 that receives a signal from a foot pedal, for example, that actuates or de-actuates a therapy component of a corresponding medical apparatus. A sense input 444 receives sensed signals from each of a number of sensing electrodes (not shown). The switches 426 selectively permit the input signal 428 (e.g., 428a and 428b) from the sense input 444 to pass through the corresponding switch 426 to the output signal 430 (e.g., 430a and 430b), or prevent the input signal 428 from the sense input 444 from passing through the corresponding switch 426.

When the foot switch is activated (e.g. closed), the switch electrically connects terminals 441a and 441b, thereby completing a current path from power supply 443 and ground 445 through pulldown resistor 447. This causes node 449 to go low. Inverter 451 inverts this signal and causes relay control node 453 to go high, thereby providing control current through the coils of normally open switches 426a and 426b. This permits the input signal 428 from the sense input 444 to pass through the corresponding switch 426 to the output signal 430 and to sensing circuitry (not shown).

When the foot switch is deactivated (e.g. opened), the switch electrically disconnects terminals 441a and 441b, thereby preventing the current path from power supply 443 to ground 445 through pulldown resistor 447. This causes node 449 to go high. Inverter 451 inverts this signal and causes relay control node 453 to go low, thereby removing control current through the coils of normally open switches 426a and 426b. This prevents the input signal 428 from the sense input 444 from passing through the corresponding switch 426 to the sensing circuitry (not shown).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A method for operating a medical apparatus that is configured to perform a therapeutic procedure on a patient, the medical apparatus includes a therapy component for performing the therapeutic procedure on the patient, the therapy component including sensing circuitry for sensing electrical signals from one or more sensing electrodes of the therapy component that are placed on and/or in the patient, the method comprising:
with the medical apparatus powered on, determining when the therapy component is actively engaged in performing the therapeutic procedure on the patient;
when the therapy component is determined to be actively engaged in performing the therapeutic procedure on the patient, having the one or more sensing electrodes of the therapy component electrically connected to the sensing circuitry of the therapy component; and
when the therapy component is determined to not be actively engaged in performing the therapeutic procedure on the patient, having the one or more sensing electrodes of the therapy component electrically isolated from the sensing circuitry of the therapy component.

2. The method of claim 1, wherein when the therapy component is determined to not be actively engaged in performing the therapeutic procedure on the patient, each of the one or more sensing electrodes is electrically isolated from the sensing circuitry by a respective relay in an open state.

3. The method of claim 2, wherein when the therapy component is determined to be actively engaged in performing the therapeutic procedure on the patient, each of the one or more sensing electrodes is electrically connected to the sensing circuitry by the respective relay in a closed state.

4. The method of claim 3, further comprising filtering each of the electrical signals that is conducted through the respective relay in the closed state before the respective electrical signal reaches the sensing circuitry.

5. The method of claim 3, wherein each of the respective relays is a normally open relay that is switched to its closed state by passing a control current though a relay coil of the relay.

6. The method of claim 5, wherein each of the respective normally open relays is high voltage relay rated for at least 5,000 volts.

7. The method of claim 1, further comprising:
closing one or more normally open relays when the therapy component is determined to be actively engaged in performing the therapeutic procedure on the patient, wherein the one or more normally open relays, when closed, electrically connect the one or more sensing electrodes to the sensing circuitry; and
opening the one or more normally open relays when the therapy component is determined to not be actively engaged in performing the therapeutic procedure on the patient, wherein the one or more normally open relays, when open, electrically isolate the one or more sensing electrodes from the sensing circuitry.

8. The method of claim 1, wherein one or more of the electrical signals are low impedance measurement signals measuring an impedance of less than 10K ohms.

9. The method of claim 8, wherein one or more of the low impedance measurement signals correspond to a clot impedance measurement signal and/or a blood coagulation measurement signal.

10. The method of claim 1, wherein the medical apparatus comprises a thrombectomy apparatus and the therapeutic procedure comprises a thrombectomy procedure.

11. The method of claim 1, further comprising activating a switch to switch between actively engaging the therapy component in performing the therapeutic procedure on the patient and not actively engaging the therapy component in performing the therapeutic procedure on the patient.

12. The method of claim 11, wherein the switch is a manually operated switch that is manually operated by an operator of the medical apparatus.

13. The method of claim 11, wherein the switch is operated by a wired or wireless control signal provided by the medical apparatus.

14. A medical apparatus for performing a therapeutic procedure on a patient, comprising:
a therapy component for performing the therapeutic procedure on the patient, the therapy component including sensing circuitry for sensing electrical signals from one or more sensing electrodes of the therapy component that are placed on and/or in the patient;
a control switch for switching between actively engaging the therapy component in performing the therapeutic procedure on the patient and disengaging the therapy component from actively performing the therapeutic procedure on the patient;
a sensing circuit protection switch operatively coupled between the one or more sensing electrodes and the sensing circuitry of the therapy component, the sensing circuit protection switch configured to:
electrically connects the one or more sensing electrodes to the sensing circuitry when the control switch actively engages the therapy component in performing the therapeutic procedure on the patient; and
electrically disconnects the one or more sensing electrodes from the sensing circuitry when the control switch disengages the therapy component from performing the therapeutic procedure on the patient.

15. The medical apparatus of claim 14, wherein the sensing circuit protection switch comprises one or more relays for selectively connecting the one or more sensing electrodes with the sensing circuitry of the therapy component when in a closed state and selectively isolating the one or more sensing electrodes from the sensing circuitry of the therapy component when in an open state.

16. The medical apparatus of claim 15, wherein the one or more relays are switched between the open state and the closed state based on a position of the control switch.

17. A defibrillation protection device for a medical apparatus, comprising:
a housing;
one or more signal inputs accessible from outside of the housing, the one or more signal inputs for receiving one or more low impedance measurement signals from one or more sensing electrodes, the low impedance measurement signals measuring an impedance of less than 10K ohms;
one or more signal outputs accessible from outside of the housing, the one or more signal outputs for passing the one or more low impedance measurement signals to a sensing circuit of the medical apparatus;
a control input;
a defibrillation protection circuit housed by the housing, the defibrillation protection circuit operatively coupled between the one or more signal inputs and the one or more signal outputs, the defibrillation protection circuit configured to:
electrically connect the one or more signal inputs to the one or more signal outputs when the control input is in a first state; and
electrically disconnect the one or more signal inputs from the one or more signal outputs when the control input is in a second state.

18. The defibrillation protection device of claim 17, wherein the control input corresponds to a manually operated switch.

19. The defibrillation protection device of claim 17, wherein the control input corresponds to a wired or wireless signal provided by the medical apparatus indicating when the medical apparatus is actively engaged in performing a therapeutic procedure on a patient.

20. The defibrillation protection device of claim 17, wherein the defibrillation protection circuit comprises one or more relays, wherein the one or more relays are closed when the control input is in the first state to electrically connect the one or more signal inputs to the one or more signal outputs, and the one or more relays are opened when the control input is in the second state to electrically disconnect the one or more signal inputs from the one or more signal outputs.
